(19) United States
(12) Patent Application Publication
(10) Pub. No.: US 2002/0091133 A1
(43) Pub. Date: Jul. 11, 2002

(54) Use of 9-substituted purine analogues and other molecules to stimulate neurogenesis

(75) Inventor: Eve M. Taylor, Del Mar, CA (US)

Correspondence Address: OPPENHEIMER WOLFF & DONNELLY LLP, Suite 700, 840 Newport Center Dr., Newport Beach, CA, 92660, US

(73) Assignee: Eve M. Taylor

(21) Appl. No.: 10/022,032

(22) Filed: Dec. 12, 2001

Related U.S. Application Data
(60) Provisional application No. 60/254,910, filed on Dec. 12, 2000.

(51) Int. Cl. A61K 31/522; A61K 31/519; A61K 31/404

(52) U.S. Cl. 514/263.3; 514/262.1; 514/263.35; 514/263.34; 514/418

(57) ABSTRACT

The present invention is directed to a method of inducing neurogenesis by administering to a mammal an effective quantity of a compound that induces neurogenesis, where neurogenesis includes proliferation of neural stem and progenitor cells, differentiation of these cells into neurons, and/or survival of these new neurons. In general, the compound comprises three moieties, A, L, and B, covalently linked. A can be a purine, tetrahydroindolone, or pyrimidine; L is a linker, while B is a moiety that promotes absorption of the compound. A particularly preferred compound is N4-[[3-(6-oxo-1,6-dihydropurin-9-yl)-1-oxopropyl] amino] benzoic acid (also known as AIT-082 or leteprinim potassium). Another aspect of the invention is pharmaceutical compositions for inducing neurogenesis.

### CROSS-REFERENCES

This application claims priority from Provisional Application Serial No. 60/254,910, by Eve M. Taylor, entitled “Use of 9-Substituted Purine Derivatives to Stimulate Proliferation of Stem Cells,” filed Dec. 12, 2001, the contents of which are incorporated herein in their entirety by this reference.

### BACKGROUND OF THE INVENTION

1. Field of the Invention

This invention is directed to methods of increasing neurogenesis by stimulating proliferation, differentiation, and/or survival of stem or progenitor cells in the nervous system, collectively called neural stem and progenitor cells, using 9-substituted purine analogues and other molecules, as well as to pharmaceutical compositions suitable for use with such methods.

2. General Background and State of the Art

Stem cells have the ability to divide for indefinite periods in culture and to give rise to specialized cells. The functions of stem cells are best described in the context of normal human development. Human development begins when a sperm fertilizes an egg and creates a single cell that has the potential to form an entire organism. This fertilized egg is totipotent, meaning that its potential is total. In the first hours after fertilization, this cell divides into identical totipotent cells. This means that either one of these cells, if placed in a woman's uterus, has the potential to develop into a fetus. In fact, identical twins develop when two totipotent cells separate and develop into two individual, genetically identical human beings. Approximately four days after fertilization and after several cycles of cell division, these totipotent cells begin to specialize, forming a hollow sphere of cells, called a blastocyst. The blastocyst has an outer layer of cells. Inside the hollow sphere of the blastocyst, there is a cluster of cells called the inner cell mass.

The outer layer of cells will go on to form a placenta and other supporting tissues needed for fetal development in the uterus. The cells of the inner cell mass will go on to form virtually all of the tissues of the human body. Although the cells of the inner cell mass can form virtually every type of cell found in the human body, they cannot independently form an organism because they are unable to give rise to the placenta and supporting tissues necessary for normal development in the human uterus. These inner cell mass cells are pluripotent. That is, they can give rise to many types of cells but not all types of cells necessary for fetal development. Because their potential is not total, they are not totipotent and they are not embryos. In fact, if an inner cell mass cell were placed into a woman's uterus, it would not develop into a fetus.

The pluripotent stem cells undergo further specialization into stem cells and are committed to give rise to cells that have a particular function. Examples of this include blood stem cells that give rise to red blood cells, white blood cells and platelets, skin stem cells give rise to the various types of skin cells, and neural stem cells that give rise to cells of the nervous system including neurons and glial cells. These more specialized stem cells are called multipotent. Multipotent stem cells, including neural stem and progenitor cells referred to here as neural stem and progenitor cells, are found in children and adults.

Stem cells are described in, e.g., M. Shamblott et al., “Derivation of Pluripotent Stem Cells from Cultured Human Primordial Germ Cells,” Proc. Natl. Acad. Sci. USA 95: 13726-13731 (1998), in J. Thompson et al., “Embryonic Stem Cell Lines Derived from Human Blastocysts,” Science 282:1145-1147 (1998), in F. H. Gage, “Stem Cells of the Central Nervous System,” Curr. Opin. Neurobiol. 8: 671-676 (1998), in L. S. Shihabuddin et al., “The Search for Neural Progenitor Cells: Prospects for the Therapy of Neurodegenerative Disease,” Mol. Med. Today 5: 474-480 (1999), in P. S. Eriksson et al., “Neurogenesis in the Adult Human Hippocampus,” Nature Med. 4: 1313-1317 (1998), in F. H. Gage, “Mammalian Neural Stem Cells,” Science 287:1433-1438 (2000), and in B. L. Jacobs et al., “Depression and the Birth and Death of Brain Cells,” Am. Scientist 88: 340-345 (2000). Stem cell biology is described in further detail in D. R. Marshak et al., eds., Stem Cell Biology (Cold Spring Harbor Laboratory Press, Cold Spring Harbor, N.Y., 2001) (“Marshak et al. (2001)”), ch. 1, pp.1-16, incorporated by this reference. The role of stem cells in neurogenesis is described in further detail in Marshak et al. (2001), ch. 18, pp. 399-438, incorporated herein by this reference.

The process by which neural stem cells give rise to neurons is called neurogenesis and consists of proliferation of neural stem and progenitor cells, differentiation of these cells into new neurons, and survival of the new neurons.

In adult mammals, including humans, neural stem and progenitor cells are found in the sub-ventricular zone (SVZ) and the dentate gyrus (DG) of hippocampus and these cells are continuously giving rise to new neurons. Neurons that are newly formed in the SVZ migrate along the rostral migratory stream into the olfactory bulb where they differentiate into granule and periglomerular neurons. In the hippocampus, neural stem and progenitor cells move into the granule layer of the hippocampus and differentiate into granule neurons. In addition to the neural stem and progenitor cells found in the dentate gyrus and subventricular zone, other regions of the nervous system contain quiescent neural stem and progenitor cells that can give rise to new neurons in cell culture conditions and yet do not in the healthy adult brain.

A number of factors have been shown to modulate neurogenesis in adult mammals. Learning and environment enrichment have been shown to enhance survival of newly born neural stem cells whereas stress has been shown to diminish proliferation of stem cells. In animal models of epilepsy and stroke proliferation of stem cells is enhanced. At the biochemical level, a number of molecules have been shown to influence neurogenesis. The growth factors EGF, bFGF and TGFβ have been shown to stimulate neurogenesis while high levels of corticosterone diminish neurogenesis.

Neuronal loss is a feature of, among others, Alzheimer's disease, Parkinson's disease, amyotrophic lateral sclerosis, also known as Lou Gehrig's disease, stroke, multiple sclerosis, traumatic brain injury, and spinal cord injury. A supply of new neurons through increased neurogenesis (i.e. increased proliferation, differentiation, and/or survival of neural stem and progenitor cells) may provide a mechanism by which disease- or injury-induced loss of neurons is ameliorated. To date, there is no method for replacing these lost neurons. Accordingly, there is a need for methods of stimulating neurogenesis. One method for doing this is by the use of pharmaceutically or biologically active compounds. Preferably, these methods should be able to be combined with methods that enable active compounds to pass through the blood-brain barrier, making therapy more efficient.

### INVENTION SUMMARY

One aspect of the present invention is a method of inducing neurogenesis, including increased proliferation, differentiation, and/or survival of neural stem and progenitor cells, comprising administering to a mammal an effective amount of a compound having activity in inducing neurogenesis, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L wherein B is -OZ or N(Y₁)-D, where Z is hydrogen, alkyl, aryl, heteroaryl, cycloalkyl, aralkyl, or heteroaralkyl; D is a moiety that promotes absorption of the compound having activity in inducing neurogenesis; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S or an effective amount of a salt or prodrug ester of the compound having activity in inducing neurogenesis.

The mammal can be an adult mammal.

In one alternative, the neural stem and progenitor cells are selected from, but not limited to, the group consisting of the neural stem and progenitor cells of dentate gyrus in the hippocampus and the neural stem and progenitor cells of the subventricular zone.

Typically, the compound is capable of bypassing the blood-brain barrier.

When moiety A is a purine moiety, it can be, but is not limited to, hypoxanthine or guanine. A can also be another naturally occurring or synthetic substituted or unsubstituted purine moiety.

When moiety A is a tetrahydroindolone moiety, it can be tetrahydroindolone or a tetrahydroindolone where oxygen is replaced by sulfur.

When moiety A is a pyrimidine moiety, it can be one of a number of naturally-occurring or synthetic pyrimidines, including, but not limited to, cytosine, thymine, uracil, or another naturally-occurring or synthetic purine.

Typically, L has the structure —(CH₂)ₙ—CONH— where n is an integer from 1 to 6.

Typically, D is a moiety having at least one polar, charged, or hydrogen-bond-forming group to increase the water-solubility of the compound having activity in inducingneurogenesis.

Another aspect of the present invention is pharmaceutical compositions. In general, a pharmaceutical composition according to the present invention comprises:

(1) a quantity of a compound effective to neurogenesis or a salt or prodrug ester of a compound effective to induce neurogenesis, the compound comprising: (a) a moiety A selected from the group consisting of a purine moiety, a purine analogue, a tetrahydroindolone moiety, a tetrahydroindolone analogue, a pyrimidine moiety, and a pyrimidine analogue; (b) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (c) a moiety B that is linked to the moiety L wherein B is -OZ or N(Y₁)-D, where Z is hydrogen, alkyl, aryl, heteroaryl, cycloalkyl, aralkyl, or heteroaralkyl; D is a moiety that promotes absorption of the compound having activity in inducing neurogenesis; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S; and

(2) a pharmaceutically acceptable carrier.

The composition is formulated for administration to a mammal to induce neurogenesis.

### BRIEF DESCRIPTION OF THE DRAWINGS

The following invention will become better understood with reference to the specification, appended claims, and accompanying drawings where:

FIG. 1 is a graph showing the time line for the experiment for which the results are presented in Example 1, showing the relative times of addition of AIT-082, BrdU and perfusion;

FIG. 2 is a graph showing the combined results of two separate experiments described in Example 1;

FIG. 3 is a graph showing the time line for the experiment for which the results are presented in Example 2, showing the relative times of addition of AIT-082, BrdU and perfusion;

FIG. 4 is a series of photomicrographs of the dentate gyrus showing immunofluorescent colocalization of cell markers (A: immunofluorescent labeling of the dentate gyrus; B-D: colocalization of BrdU and NeuN; E-G: colocalization of BrdU and S100β); and

FIG. 5 is a series of graphs showing the BrdU-positive cells in dentate gyrus as a function of the dose of AIT-082 (A: total BrdU-positive cells; B: BrdU-positive cells broken down into neurons, astrocytes, and other cells).

### DETAILED DESCRIPTION OF THE PREFERRED EMBODIMENTS

I have discovered that the purine derivative N-4-[[3-(6-oxo-1,6-dihydropurin-9-yl)-1-oxopropyl] amino] benzoic acid (also known as AIT-082 and leteprinim potassium), which passes through the blood-brain barrier, can stimulate neurogenesis. Other compounds, including other purine analogues and derivatives, pyrimidine analogues and derivatives, and tetrahydroindolone analogues and derivatives, are also expected to have this activity and are useful in methods according to the present invention.

Accordingly, in general, a method according to the present invention of inducing neurogenesis comprises administering to a mammal an effective quantity of a compound, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L though a carbonyl group wherein B is -OZ or N(Y₁)-D, where Z is hydrogen, alkyl, aryl, heteroaryl, cycloalkyl, aralkyl, or heteroaralkyl; D is a moiety that promotes absorption of the compound having activity in inducing neurogenesis; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

Typically, a compound useful in a method of the present invention is capable of passing through the blood-brain barrier.

In one preferred embodiment of methods according to the present invention, the moiety A is a purine moiety.

In one alternative, A is a substituted or unsubstituted hypoxanthine moiety. Typically, in this alternative, L has the structure —(CH2)ₙ where n is an integer from 1 to 6.

The compound having the activity of inducing neurogenesis can be a compound of formula (I)

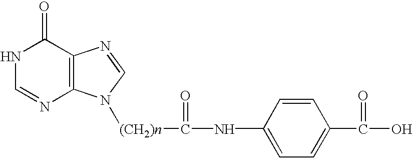

where n is an integer from 1 to 6 and R is hydrogen or lower alkyl or is a salt or prodrug ester of a compound of formula (I) wherein n is an integer from 1 to 6 and R is hydrogen or lower alkyl. Typically, the compound is a compound of formula (I) wherein n is an integer from 1 to 6 and R is hydrogen or lower alkyl. Typically, R is hydrogen, and the compound is N-4-[[3-(6-oxo-1,6-dihydropurin-9-yl)-1-oxopropyl] amino] benzoic acid, designated AIT-082. Alternatively, R is ethyl, and the compound is N-4-[[3-(6-oxo-1,6-dihydropurin-9-yl)-1-oxopropyl] amino] benzoic acid ethyl ester. The activity of this compound is described further in the Examples.

Alternatively, the purine derivative can be a 9-substituted hypoxanthine derivative of formula (II)

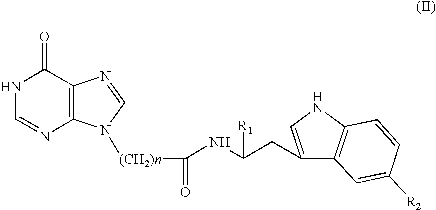

wherein n is a integer from 1 to 6, R₁ is selected from the group consisting of H, COOH, and COOW₁, where W₁ is selected from the group consisting of lower alkyl, amino, and lower alkylamino, and R₂ is selected from the group consisting of H and OH.

In this alternative, for one particularly preferred purine derivative, n is 2, R₁ is H and R₂ is OH and the purine derivative is N-(2-(5-hydroxyindol-3-yl))ethyl-3-(6-oxohydropurine-9-yl) propanamide. In this alternative, for another particularly preferred purine derivative, n is 2, R₁ is H and R₂ is H and the purine derivative is N-(2-indol-3-yl)ethyl-3-(6-oxohydropurin-9-yl) propanamide. In this alternative, for still another particularly preferred purine derivative, n is 2, R₁ is COOH, and R₂ is OH and the purine derivative is N-(1-carboxyl-(2-(5-hydroxyindol-3-yl))ethyl-3-(6-oxohydropurin-9-yl) propanamide.

As another alternative, the purine derivative can be a 9-substituted hypoxanthine derivative of formula (III)

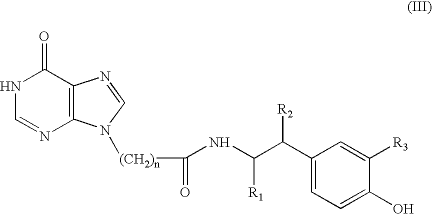

wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting of H and OH.

In this alternative, for one particularly preferred purine derivative, n is 2, R₁ is H, R₂ is H, and R₃ is OH, and the purine derivative is N-(2-(3,4-dihydroxyphenyl))ethyl-3-(6-oxohydropurin-9-yl) propanamide. In this alternative, for another particularly preferred purine derivative, n is 2, R₁ is H, R₂ is OH, and R₃ is OH, and the purine derivative is N-(2-hydroxy-2-(3,4-dihydroxyphenyl))ethyl-3-(6-oxohydropurin-9-yl) propanamide. In this alternative, for still another particularly preferred purine derivative, n is 2, R₁ is COOH, R₂ is H, and R₃ is OH, and the purine derivative is N-(1-carboxyl-2-(3,4-dihydroxyphenyl))ethyl-3-(6-oxohydropurin-9-yl) propanamide.

When the purine moiety is guanine, one preferred purine derivative is a 9-substituted guanine derivative of formula (IV)

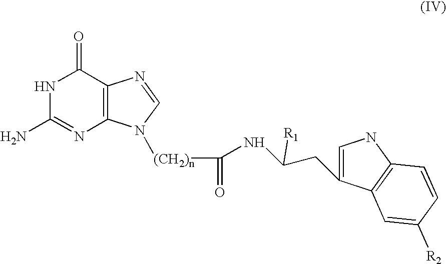

wherein n is an integer from 1 to 6, R₁ is selected from the group consisting of H, COOH, and COOW₁, or W₁ is lower alkyl, amino, or lower alkylamino, and R₂ is selected from the group consisting of H and OH.

In this alternative, for one particularly preferred purine derivative, n is 2, R₁ is H, and R₂ is OH, and the purine derivative is N-(2-(5-hydroxindol-3-yl))ethyl-3-(2-amino-6-oxohydropurin-9-yl) propanamide. In this alternative, for another particularly preferred purine derivative, n is 2, R₁ is H, and R₂ is H and the purine derivative is N-(2-(2-indol-3-yl)ethyl))-3-(2-amino-6-oxohydropurin-9-yl)) propanamide. In this alternative, for still another particularly preferred purine derivative, n is 2, R₁ is COOH, and R₂ is OH, and the purine derivative is N-(1-carboxyl)-(2-(5-hydroxyindol-3-yl))ethyl-3-(2-amino-6-oxohydropurin-9-yl) propanamide.

Alternatively, the purine derivative can be a 9-substituted guanine derivative of formula (V) wherein n is an integer from 1 to 6.

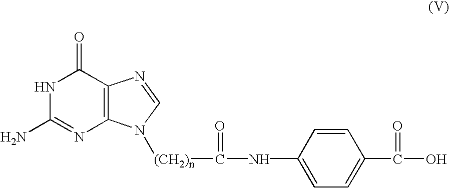

In this alternative, for one particularly preferred purine derivative, n is 2 and the compound is N-4-carboxyphenyl-3-(2-amino-6-oxohydropurin-9-yl) propanamide.

Alternatively, the purine derivative can be a 9-substituted guanine derivative of formula (VI) wherein n is an integer from 1 to 6.

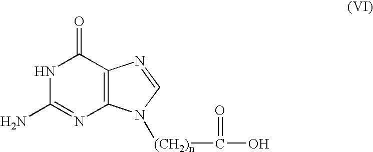

In this alternative, for one particularly preferred purine derivative, n is 2 and the compound is 3-(2-amino-6-oxohydropurine-9-yl) propanoric acid.

Alternatively, the purine derivative can be a 9-substituted guanine derivative of formula (VII) wherein n is an in integer from 1 to 6, p is an integer from 1 to 6, and q is an integer from 1 to 3.

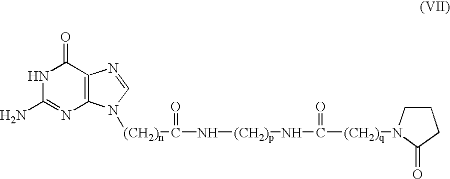

In this alternative, for one particularly preferred purine derivative, n is 2, p is 2, and q is 1, and the purine derivative is N-[2-[[2-(2-oxopyrrolidin-1-yl)-1-oxoethyl]amino]ethyl] propanamide.

Alternatively, the purine derivative can be a 9-substituted guanine derivative of formula (VIII) wherein R₁ is selected from the group consisting of H, COOH, and COOW₁, where W₁ is selected from the group consisting of lower alkyl, amino, and lower alkylamino, R₂ is selected from the group consisting of H and OH, and R₃ is selected from the group consisting of H and OH.

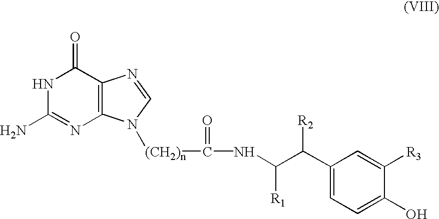

In this alternative, for one particularly preferred purine derivative, n is 2, R₁ is H, R₂ is H, and R₃ is OH, and the purine derivative is N-(2-(3,4-dihydroxyphenyl)ethyl-3-(2-amino-6-oxohydropurin-9-yl) propanamide. In this alternative, for another particularly preferred purine derivative, n is 2, R₁ is H, R₂ is OH, and R₃ is OH, and the purine derivative is N-(2-hydroxy-2-(3,4-dihydroxyphenyl)ethyl)-3-(2-amino-6-oxohydropurin-9-yl) propanamide. In this alternative, for still another particularly preferred purine derivative, n is 2, R₁ is COOH, R₂ is H, and R₃ is H and the compound is N-(1-carboxyl-2-(3,4-dihydroxyphenyl)ethyl)-3-(2-amino-6-oxohydropurin-9-yl) propanamide.

Alternatively, the purine derivative can be a 9-substituted guanine derivative of formula (IX) wherein n is an integer from 1 to 6 and p is an integer from 1 to 3.

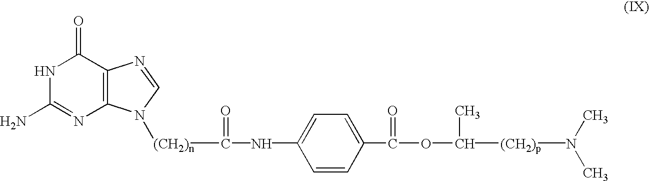

In this alternative, for one particularly preferred purine derivative, n is 2, p is 1, and the compound is the 1-(dimethylamino)-2-propyl ester of N-4-carboxyphenyl-3-(2-amino-6-oxohydropurin-9-yl) propanamide.

Other bifunctional hypoxanthine derivatives suitable for use in methods according to the present invention are disclosed in U.S. Pat. No. 5,091,432 to Glasky, incorporated herein by this reference. Other bifunctional guanine derivatives suitable for use in methods according to the present invention are disclosed in U.S. Pat. No. 6,297,226 to Glasky, incorporated herein by this reference.

More generally, purine-based compounds suitable for use in methods according to the present invention are compounds in which A is a substituted or unsubstituted 9-atom bicyclic moiety in which the 5-membered ring has 1 to 3 nitrogen atoms, the bicyclic moiety having the structure of formula (X)

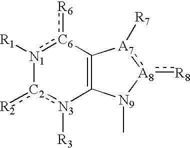

where:

(1) if the bond between N₁ and the bond between C₅ is a single bond, then the bond between C₆ and R₆ is a double bond, R₆ is O or S, and R₁ is hydrogen, alkyl, aralkyl, cycloalkyl, or heteroaralkyl;

(2) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(3) if the bond between C₂ and N₃ is a single bond, then the bond between C₂ and R₂ is a double bond, R₂ is O or S, and R₃ is hydrogen or alkyl;

(4) if the bond between C₂ and N₃ is a double bond, then the bond between C₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(5) A₇ and A₈ are C or N;

(a) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;

(b) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;

(c) if A₇ and A₈ are both N, then the bond between A₇ and A₈ is a double bond, and R₇ and R₈ are not present;

(d) if A₇ is C and A₈ is N, then the bond between A₇ and A₈ is a double bond, R₇ is hydrogen, and R₈ is not present;

(e) if A₇ is N, A₈ is C, and the bond between A₇ and A₈ is a double bond, then R₇ is not present, the bond between A₈ is a single bond, and R₈ is hydrogen, halo, alkyl, alkenyl, aryl, aralkyl, aralkenyl, heteroaryl, heteroaralkyl, or heteroaralkenyl;

(f) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S; and

(6) N₉ is bonded to L; with the proviso that A does not have the structure of an unsubstituted guanine or hypoxanthine.

The purine moiety can be a purine moiety of formula (XI)

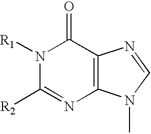

in which:

(1) R₁ is selected from the group consisting of hydrogen, alkyl, aralkyl, cycloalkyl, and heteroaralkyl; and

(2) R₂ is selected from the group consisting of hydrogen, alkyl, aralkyl, cycloalkyl, heteroaralkyl, halo, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroarylkylaminocarbonyl in which the alkyl portions could be cyclic and can contain from one to three heteroatoms which could be N, O, or S, with the proviso that both R₁ and R₂ are not hydrogen and that R₁ is not hydrogen when R₂ is amino.

The purine moiety of formula (XI) is a hypoxanthine or a guanine derivative but excludes unsubstituted hypoxanthine, in which R₁ and R₂ are hydrogen, and unsubstituted guanine, in which R₁ is hydrogen and R₂ is amino.

In one particularly preferred embodiment, R₁ is butyl and R₂ is hydrogen.

In another preferred embodiment, R₁ is benzyl and R₂ is hydrogen.

In another preferred embodiment, R₁ is dimethylaminoethyl and R₂ is hydrogen.

In another preferred embodiment, R₁ is cyclopentyl and R₂ is hydrogen.

In another preferred embodiment, R₁ is cyclohexylmethyl and R₂ is hydrogen.

In another preferred embodiment, R₁ is cyclopropylmethyl and R₂ is hydrogen.

In another preferred embodiment, R₁ is hydrogen and R₂ is phenyl.

In another preferred embodiment, R₁ is hydrogen and R₂ is trifluoromethyl.

In another preferred embodiment, R₁ is hydrogen and R₂ is butyl.

In another preferred embodiment, R₁ is butyl and R₂ is butyl.

In another preferred embodiment, R₁ is hydrogen and R₂ is methyl.

In another preferred embodiment, R₁ is hydrogen and R₂ is phenylamino.

Alternatively, the purine moiety is a purine moiety of Formula (XII)

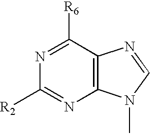

in which:

(1) R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃ where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaryloxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

(2) R₆ is selected from the group consisting of hydrogen, halo, amino, OQ₅, SQ₅, NHNH₂, NHOQ₅, NQ₅Q₆, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl in which the alkyl portions can be cyclic and can contain from 1 to 3 heteroatoms which can be N, O, or S.

In one preferred example of this embodiment, R₂is hydrogen and R₆ is —NH₂ or —N(CH₃)₂.

In another preferred example of this embodiment, R₂ is hydrogen and R₆ is Cl.

In yet another preferred example of this embodiment, R₂ is —NH₂ and R₆ is Cl.

In another alternative, the purine moiety is the purine moiety of Formula (XIII)

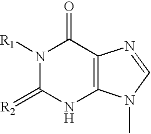

in which:

(1) R₁ is hydrogen, alkyl, aralkyl, cycloalkyl, or heteroaralkyl; and

(2) R₂is O or S.

Preferably, in this embodiment, R₁ is hydrogen and R₂ is O or S.

Particularly preferred purine-based compounds for use in methods according to the present invention include: (1) 4-[3-(1-benzyl-6-oxo-1,6-dihydropurin-9-yl)propionylamino] benzoic acid ethyl ester; (2) 4-[3-(1-butyl-6-oxo-1,6-dihydropurin-9-yl)propionylamino] benzoic acid ethyl ester; (3) 4-[3-(1-methyl-6-oxo-1,6-dihydropurin-9-yl)propionylamino] benzoic acid ethyl ester; (4) 4-[3-(1-(2dimethylaminoethyl)-6-oxo-1,6-dihydropurin-9-yl)propionylamino] benzoic acid ethyl ester; (5) 4-[3-(2,6-dioxo-1,2,3,6-tetrahydropurin-9-yl)propionylamino] benzoic acid ethyl ester; (6) 4-[3-(6-methoxypurin-9-yl)propionylamino] benzoic acid ethyl ester; (7) 4-[3-(6-dimethylaminopurin-9-yl)propionylamino] benzoic acid ethyl ester; (8) 4-[3-(2-amino-6-chloropurin-9-yl)propionylamino] benzoic acid ethyl ester; (9) 4-[2-(6-oxo-2-thioxo-1,2,3,6-tetrahydropurin-9-yl)propionylamino]benzoic acid ethyl ester; (10) 4-[2-(2-butyl-6-oxo-1,6-dihydropurin-9-yl)propionylamino]benzoic acid ethyl ester; (11) 4-[2-(6-oxo-2-phenyl-1,6-dihydropurin-9-yl)propionylamino]benzoic acid ethyl ester; (12) 4-{[3-(6-chloropurin-9-yl)propionyl]methylamino} benzoic acid methyl ester; (13) 3-(1-benzyl-6-oxo-1,6-dihydropurin-9-yl)-N-[3-(2-oxopyrrolidin-1-yl)propyl] propionamide; (14) 3-(1-benzyl-6-oxo-1,6-dihydropurin-9-yl)-N-{2-[2-(2-oxopyrrolidin-1-yl)acetylamino]ethyl} propionamide; (15) N-3-(2-oxopyrrolidin-1-yl)propyl]-3-(6-oxo-2-thioxo-1,2,3,6-tetrahydropurin-9-yl) propionamide; and (16) 3-(1-benzyl-6-oxo-1,6-dihydropurin-9-yl)-N-(3-morpholin-4-yl-propyl) propionamide.

In another alternative of methods according to the present invention, the compound is a tetrahydroindolone derivative or analogue where A is a 9-atom bicyclic moiety in which the 5-membered ring has one to three nitrogen atoms, the bicyclic moiety having the structure of formula (XIV)

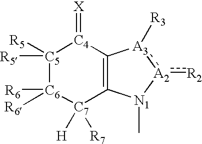

where:

(1) N₁ is bonded to L;

(2) A₂and A₃are C or N;

(a) If A₂ and A₃ are both C and the bond between A₂ and A₃ is a single bond, then the bond between A₂ and R₂ is two single bonds, two hydrogen atoms or is a double bond in which R₂ is O or S and R₃ is two hydrogen atoms;

(b) If A₂ and A₃ are both C and the bond between A₂ and A₃ is a double bond, then R₃ is hydrogen, the bond between A₂ and R₂ is a single bond and R₂ is hydrogen, halo, alkyl, alkenyl, aryl, aralkyl, aralkenyl, heteroaryl, heteroaralkyl, or heteroaralkenyl;

(c) If A₂ and A₃ are both N, then the bond between A₂ and A₃ is a double bond and R₂ and R₃ are not present;

(d) If A₂ is N and A₃ is C, then the bond between A₂ and A₃ is a double bond, R₂ is not present, and R₃ is hydrogen;

(e) If A₂ is C, A₃ is N, and the bond between A₂ and A₃ is a double bond, then R₃ is not present, the bond between A₂ and R₂ is a single bond, and R₂ is hydrogen, halo, alkyl, alkenyl, aryl, aralkyl, aralkenyl, heteroaryl, heteroaralkyl, or heteroaralkenyl;

(f) If A₂ is C, A₃ is N, and the bond between A₂ and A₃ is a single bond, then R₃ is hydrogen, alkyl, aryl, aralkyl, heteroaryl, or heteroaralkenyl, the bond between A₂ and R₂ is a double bond, and A₂ is O or S;

(3) R₅ is hydrogen, alkyl, aryl, aralkyl, heteroaryl, heteroaralkyl, alkanoyl, aroyl, heteroaroyl, aralkanoyl, heteroaralkanoyl, NH₂, NHQ₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(4) R_{5′} is hydrogen unless R₅ is alkyl, in which case R₅ is hydrogen or the same alkyl as R₅;

(5) R₅ and R_{5′} can be taken together as a double bond to C₅, and can be O, S, NQ₃, or C which can be substituted with one or two groups R₅, where Q₃ is alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S;

(6) R₆ is hydrogen, alkyl, aryl, aralkyl, heteroaryl, heteroaralkyl, NH₂, NHQ₄, NQ₄Q₅, OH, OQ₄, or SQ₄, where Q₄ and Q₅ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₄ and Q₅ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(7) R_{6′} is hydrogen unless R₆ is alkyl, in which case R_{6′} is hydrogen or the same alkyl as R₆;

(8) R₆ and R_{6′} can be taken together as a double bond to C₆ and can be O, S, NQ₆, or C which can be substituted with one or two groups R₅, and where Q₆ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S;

(9) R₇ is hydrogen unless R₅ is alkyl and R_{5′} is hydrogen, in which case R₇ is the same alkyl as R_{5·}; and

(10) X is oxygen, sulfur, or NH.

Typically, A is a tetrahydroindolone moiety. More typically, the tetrahydroindolone moiety is a tetrahydroindolone moiety of formula (XV)

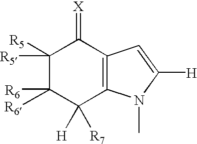

in which:

(1) R₅ is hydrogen, alkyl, aryl, aralkyl, heteroaryl, heteroaralkyl, alkanoyl, aroyl, heteroaroyl, aralkanoyl, heteroaralkanoyl, NH₂, NH₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S;

(2) R_{5′} is hydrogen;

(3) R₆ is hydrogen, alkyl, aryl, aralkyl, heteroaryl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S and where W₁ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from one to three heteroatoms which can be N, O, or S;

(4) R_{6′} is hydrogen;

(5) R₇ is hydrogen; and

(6) X is oxygen, sulfur, or NH.

Typically, R₅, R_{5′}, R₆, R_{6′}, and R₇ are all hydrogen.

When A is a tetrahydroindolone moiety, preferred compounds are 4-[3-(4-oxo-4,5,6,7-tetrahydroindolon-1-yl) propionylamino] benzoic acid ethyl ester and 4-[3-(4-oxo-4,5,6,7-tetrahydroindolon-1-yl) propionylamino] benzoic acid.

In another alternative, the compound is a pyrimidine derivative or pyrimidine analogue. In this alternative, A is an amino-substituted 6-membered heterocyclic moiety of formula (XVI)

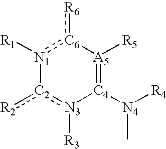

where:

(1) if the bond between N₁ and the bond between C₆ is a single bond, then the bond between C₆ and R₆ is a double bond, R₆ is O or S, and R₁ is hydrogen, alkyl, aralkyl, cycloalkyl, or heteroaralkyl;

(2) if the bond between N₁ and C₆ is a double bond, then the bond between C₆ and R₆ is a single bond, R₁ is not present, and R₆ is hydrogen, halo, amino, OH, OQ₁, SQ₁, NHNH₂, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(3) if the bond between C₂ and N₃ is a single bond, then the bond between C₂ and R₂ is a double bond, R₂ is O or S, and R₃ is hydrogen or alkyl;

(4) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OH, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(5) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;

(6) A₅ is carbon or nitrogen;

(7) if A₅ is nitrogen, then R₅ is not present;

(8) if A₅ is carbon, then R₅ is hydrogen, amino, alkyl, alkoxy, halo, nitro, aryl, cyano, alkenyl, or alkaryl;

(9) if R₅ and R₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

(10) N₄ is bonded to L.

Typically, A₅ is carbon and the 6-membered heterocyclic moiety is a pyrimidine moiety.

When A is a pyrimidine moiety, in one alternative, R₂ is O and R₃ is hydrogen. In this alternative, the pyrimidine moiety can be cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, or 5-hydroxymethyluracil.

In another alternative, R₂ is S and R₃ is hydrogen. In this alternative, the pyrimidine moiety can be 2-thiouracil, 5-methylamino-2-thiouracil, 5-methyl-2-thiouracil, or 2-thiocytosine.

In still another alternative, R₂ is amino and the bond between C₂ and N₃ is a double bond. In this alternative, the pyrimidine moiety can be 2-aminopyrimidinone or 2-amino-4-chloropyrimidine.

In still another alternative, R₂ is hydrogen and the bond between C₂ and N₃ is a double bond. In this alternative, the pyrimidine moiety can be 4-chloropyrimidine, 5-amino-4-chloropyrimidine, 4-chloro-5-methylpyrimidine, 4-chloro-5-hydroxymethylpyrimidine, or 4-chloro-5-carboxymethylpyrimidine.

In still another alternative, R₁ is hydrogen, methyl, or ethyl, R₅ is hydrogen, methyl, or ethyl, and R₆ is O. In this alternative, the pyrimidine moiety can be pyrimidinone.

Particularly preferred pyrimidine compounds include: 4-[3-(2-amino-6-chloropyrimidin-4-ylamino) propionylamino] benzoic acid ethyl ester; 4-[3-(5-amino-6-chloropyrimidin-4-ylamino) propionylamino] benzoic acid ethyl ester; 4-[3-(6-chloropyrimidin-4-ylamino) propionylamino] benzoic acid ethyl ester; 4-[3-(2-amino-6-chloropyrimidin-4-ylamino) propionylamino] benzoic acid; 4-[3-(6-chloropyrimidin-4-ylamino) propionylamino] benzoic acid; 4-[3-(5-amino-6-chloropyrimidin-4-ylamino) propionylamino] benzoic acid; 3-[3-(2-amino-6-chloropyrimidin-4-ylamino) propionylamino] benzoic acid ethyl ester; 3-[3-(6-chloropyrimidin-4-ylamino) propionylamino] benzoic acid ethyl ester; 3-[3-(5-amino-6-chloropyrimidin-4-ylamino) propionylamino] benzoic acid ethyl ester; 3-[3-(2-amino-6-chloropyrimidin-4-ylamino) propionylamino] benzoic acid; 3-[3-(6-chloropyrimidin-4-ylamino) propionylamino] benzoic acid; and 3-[3-(5-amino-6-chloropyrimidin-4-ylamino) propionylamino] benzoic acid.

In accordance with the present invention, and as used herein, the following terms, when appearing alone or as part of a moiety including other atoms or groups, are defined with the following meanings, unless explicitly stated otherwise. In addition, all groups described herein can be optionally substituted unless such substitution is excluded. The term “alkyl,” as used herein at all occurrences, refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “alkenyl,” as used herein at all occurrences, refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “alkoxy” refers to the ether —O-alkyl, where alkyl is defined as above. The term “aryl” refers to aromatic groups that have at least one ring having a conjugated π-electron system and includes carbocyclic aryl and biaryl, both of which may be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, isothiazole, pyrazole, pyrrole, each of which can be optionally substituted as discussed above. The term “sulfonyl” refers to the group —S(O₂)—. The term “alkanoyl” refers to the group —C(O)Rg, where Rg is alkyl. The term “aroyl” refers to the group —C(O)Rg, where Rg is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group. As used herein, the term “lower,” in reference to an alkyl or the alkyl portion of another group including alkyl, is defined as a group containing one to six carbon atoms. The term “optionally substituted” refers to one or more substituents that can be lower alkyl, aryl, amino, hydroxy, lower alkoxy, aryloxy, lower alkylamino, arylamino, lower alkylthio, arylthio, or oxo, in some cases, other groups can be included, such as cyano, acetoxy, or halo. The term “halo” refers generally to fluoro, chloro, bromo, or iodo; more typically, “halo” refers to chloro.

As indicated above, the linker L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo. Preferably, the linker L has the structure —(CH₂)ₙ— wherein n is an integer from 1 to 6. As detailed below, for most preferred embodiments of compounds useful in methods according to the present invention, a preferred linker has n equal to 2 or 3.The moiety B is either: (i) -OZ, where Z is hydrogen, alkyl, aryl, heteroaryl, cycloalkyl, aralkyl, or heteroaralkyl; or (ii) N(Y₁)-D, where D is a moiety that promotes absorption of the compound, and Y₁ is hydrogen, alkyl, aryl, heteroaryl, aralkyl, heteroaralkyl, which, when taken with D, can form a cyclic 5- or 6-membered saturated ring which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen. Where the moiety B is -OZ, the moiety B is a carboxylic acid or carboxylic acid or ester. Typically, where B is a carboxylic acid ester, the moiety Z is a lower alkyl, such as methyl, ethyl, butyl, propyl, or isopropyl.

In one alternative, the moiety D, as described above, is a moiety having at least one polar, charged, or hydrogen-bond-forming group to improve the metabolic and bioavailability properties of the compound. The moiety D can be, but is not limited to, a moiety with physiological or biological activity such as nootropic activity. In one alternative, the moiety D can be a moiety containing at least one carboxyl, carboxamide, carboxyl ester, or carbonyl function. In another alternative, the moiety D can be a moiety containing at least one hydroxyl, primary amino, secondary amino, tertiary amino, sulfhydryl, or sulfonamidyl function. The moiety D can be cyclic or acyclic. Preferred examples of the moiety D are described below.

When the moiety D is a cyclic or acyclic moiety containing at least one carbonyl, carboxamide, carboxyl ester, or carbonyl function, in one preferred example, D is a carboxylic acid or carboxylic acid ester with the structure

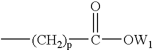

wherein p is an integer from 1 to 6 and W₁ is selected from the group consisting of hydrogen and lower alkyl. Typically, if W₁ is lower alkyl, it is methyl, ethyl, propyl, butyl, or isobutyl. Typically, p is 3. Typically, W₁ is hydrogen or ethyl.

In another preferred example, D and Y₁ are taken together to form a piperazine derivative as described in D. Manefti et al., “Molecular Simplification of 1,4-Diazabicyclo[4.3.0]nonan-9-ones Gives Piperazine Derivatives That Maintain High Nootropic Activity,” J. Med. Chem. 43: 4499-4507 (“Manetti et al. (2000)”). B is an analogue of structure

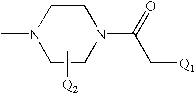

wherein Q₁ is hydrogen, methyl, ethyl, butyl, or propyl, Q₂ is hydrogen or methyl, where, if Q₂ is methyl, it can be located at either of the two possible positions in the piperazine ring.

In another preferred example, D has the structure

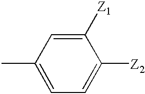

where one of Z₁ and Z₂ is hydrogen, and the other of Z₁ and Z₂ is —COOH or —COOW₁, wherein W₁ is alkyl. Typically, W₁ is selected from the group consisting of methyl, ethyl, propyl, butyl, and isobutyl. Either of Z₁ or Z₂ can be hydrogen. When Z₁ is hydrogen and Z₂ is —COOH, the moiety B is p-aminobenzoic acid (PABA). When Z₁ is —COOH and Z₂ is hydrogen, the moiety B is m-aminobenzoic acid (MABA). When Z₁ is hydrogen and Z₂ is —COOW₁, the moiety B is an ester of p-aminobenzoic acid (PABA). When Z₁ is —COOW₁ and Z₂ is hydrogen, the moiety B is an ester of m-aminobenzoic acid (MABA). Typically, these esters are ethyl esters.

When the moiety D is a moiety that contains at least one hydroxyl, primary amino, secondary amino, tertiary amino, sulfhydryl, or sulfonamidyl function, in one preferred example, D is a phenylsulfonamidyl moiety of structure

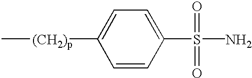

wherein p is an integer from 0 to 6. Typically, p is 2.

In another preferred example, D is an alkylpyridyl moiety of structure

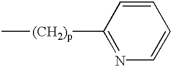

wherein p is an integer from 1 to 6. Typically, p is 1.

In another preferred example, D is a dialkylaminoalkyl moiety of the structure

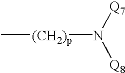

wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Where Q₇ and Q₈ can be taken together to form a five or six member ring, the ring is typically pyrrolidine, piperidine, or morpholine. The pyrrolidine ring can be optionally substituted with oxo. The piperidine ring can be optionally substituted with methyl or ethyl. Typically, p is 2 or 3.

In another preferred example, D is an alkylpyrrolidine moiety of the structure

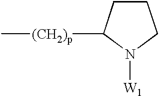

wherein p is an integer from 1 to 6 and W₁ is selected from the group consisting of methyl, ethyl, and propyl. Typically, W₁ is methyl. Typically, p is 2.

Preferably, a compound useful in methods according to the present invention has a log P of from about 1 to about 4 in order to optimize bioavailability and CNS penetration of the compound.

In general, compounds that are suitable for methods according to the present invention also include salts and prodrug esters of these compounds. It is well known that purines, purine analogues, pyrimidines, pyrimidine analogues, tetrahydroindolones, and tetrahydroindolone analogues have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound in methods according to the present invention or in pharmaceutical compositions suitable for methods according to the present invention therefore includes such salt forms as occur at physiological pH or at the pH of a pharmaceutical composition unless those salt forms are specifically excluded.

Similarly, prodrug esters can be formed by reaction of either a carboxyl or a hydroxyl group on the compound with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, halo, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art. The recitation of a compound in methods according to the present invention or in pharmaceutical compositions suitable for methods according to the present invention therefore includes such prodrugs unless prodrugs are specifically excluded.

The mammal can be an adult mammal.

Typically, the neural stem and progenitor cells are located in the dentate gyrus of the hippocampus and the subventricular zone. Alternatively, however, the neural stem and progenitor cells are located in other locations in which the cells are normally quiescent or substantially quiescent. Methods according to the present invention are not limited to neural stem and progenitor cells located in the dentate gyrus of the hippocampus or the subventricular zone.

Other bifunctional hypoxanthine analogues suitable for use in methods according to the present invention are disclosed in U.S. Pat. No. 5,091,432 to Glasky, incorporated herein by this reference. Other bifunctional guanine analogues suitable for use in methods according to the present invention are disclosed in U.S. Pat. No. 6,297,226, by Glasky et al., incorporated herein by this reference.

Exemplary studies and treatments were performed as discussed below using various dosages and routes of administration of a selected exemplary purine analog representative of compositions that are effective with the methods of the present invention. Of course, those skilled in the art will recognize that the present invention is not specifically limited to the particular compositions, dosages, or routes of administration detailed below.

Depending upon the particular needs of the individual subject involved, the compositions used in the present invention can be administered in varying doses to provide effective treatment concentrations based upon the teachings of the present invention. What constitutes an effective amount of the selected composition will vary based upon such factors as the activity of the selected compound, the physiological characteristics of the subject, the extent and nature of the subject's disease or condition and the method of administration. Exemplary treatment concentrations that are proven effective in stimulating neurogenesis range from less than 0.1 mg/kg to 10 mg/kg or more. Generally, initial doses will be modified to determine the optimum dosage for treatment of the particular mammalian subject. The compositions can be administered using a number of different routes including orally, topically, transdermally, administration by intraperitoneal injection or administration by intravenous injection directly into the bloodstream. Of course, effective amounts of the compounds can also be administered through injection into the cerebrospinal fluid or infusion directly into the brain, if desired.

The methods of the present invention can be affected using the compounds described above administered to a mammalian subject either alone or in combination as a pharmaceutical formulation. Further, the compounds described above can be combined with pharmaceutically acceptable excipients and carrier materials such as inert solid diluents, aqueous solutions, or non-toxic organic solvents. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art including, but not limited to, human serum albumin, ion exchangers, dextrose, alumina, lecithin, buffer substances such as phosphate, glycine, sorbic acid, potassium sorbate, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formulations (i.e., they can be “nebulized”) to be administered via inhalation. Aerosol formulations can be placed into pressurized acceptable propellants, such as dichloromethane, propane, or nitrogen. Other suitable propellants are known in the art.

Formulations suitable for parenteral administration, such as, for example, by intravenous, intramuscular, intradermal, and subcutaneous routes, include aqueous and non-aqueous, isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solubilizers, stabilizers, and preservatives. Compositions suitable for use and methods according to the present invention can be administered, for example, by intravenous infusion, orally, topically, intraperitoneally, intravesically, or intrathecally. Formulations of compounds suitable for use in methods according to the present invention can be presented in unit-dose or multi-dose sealed containers, in physical form such as ampoules or vials.

Accordingly, another aspect of the present invention is a pharmaceutical composition comprising:

(1) a quantity of a compound effective to induce neurogenesis or a salt or prodrug ester of a compound effective to induce neurogenesis, the compound can be a purine derivative or purine analogue, a pyrimidine derivative or pyrimidine analogue, or a tetrahydroindolone derivative or tetrahydroindolone analogue as described above and

(2) a pharmaceutically acceptable carrier.

The composition is formulated for administration to a mammal to induce one or aspects of neurogenesis.

The invention is illustrated by the following Examples. These Examples are presented for illustration only and are not intended to limit the invention.

### EXAMPLES

### Example 1

### Effects of the Bifunctional Purine Analogue N-4-Carboxyphenyl-3-(6-Oxohydropurin-9-yl)Propanamide in Stimulating Neural Stem and Progenitor Cell Proliferation

Experimental Design

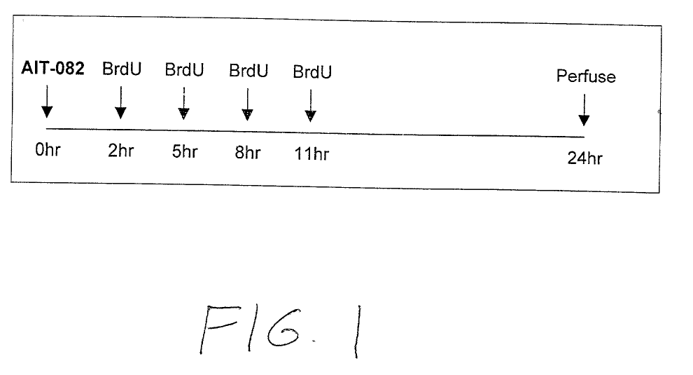

In two separate experiments, AIT-082 was administered intraperitoneally to adult mice (5-7 per group) at 0.01-100 mg/kg. Saline was given as a negative control. Starting two hours after AIT-082 administration, animals received four intraperitoneal injections of bromodeoxyuridine (BrdU; 50 mg/kg each) at 3 hr intervals. BrdU is a thymidine analogue that is incorporated into DNA during synthesis and thus labels newly formed cells. Animals were perfused at 24 hr after the AIT-082 administration. This treatment regimen is outlined in FIG. 1.

Animals were perfused transcardially with 50 mL ice-cold phosphate buffered saline (PBS) and then 100 mL of 4% paraformaldehyde in PBS. Brains were removed, post-fixed in 4% paraformaldehyde for 24 hr at 4° C., and then transferred to 30% sucrose at 4° C. for a minimum of 3 days before sectioning. Coronal sections (40 μm) were prepared using a freezing microtome and stored in cryoprotectant at −20° C. before immunostaining for BrdU.

Every sixth section through the rostral-caudal extent of the hippocampus was immunostained for BrdU with mouse anti-BrdU paired with a biotinylated goat anti-mouse IgG. Avidin-biotin-horseradish peroxidase (HRP) complex was applied to sections and immunoreactivity visualized by reacting diaminobenzidine with the HRP. Stereological techniques were used to estimate the total number of BrdU-positive cells in the dentate gyrus.

Results

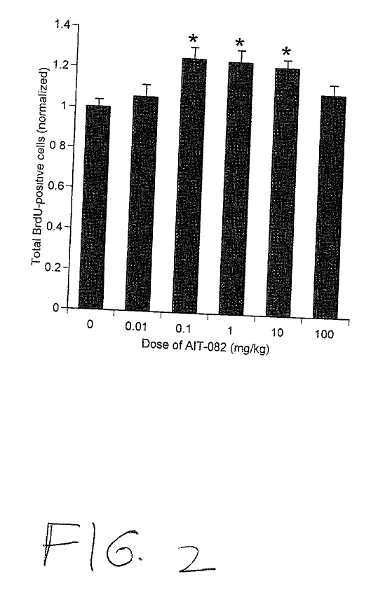

The data from each experiment was normalized to the appropriate control data and the data from both experiments combined (FIG. 2). There was a statistically significant increase in the total number of BrdU-positive cells in dentate gyrus in mice treated with 0.1-10 mg/kg AIT-082.

Conclusion

These data indicate that a single administration of AIT-082 increased the proliferation of neural stem and progenitor cells in the dentate gyrus.

### Example 2

### Effects of the Bifunctional Purine Analogue N-4-Carboxyphenyl-3-(6-Oxohydropurin-9-yl)Propanamide in Stimulating Neural Stem and Progenitor Cell Differentiation

Experimental Design

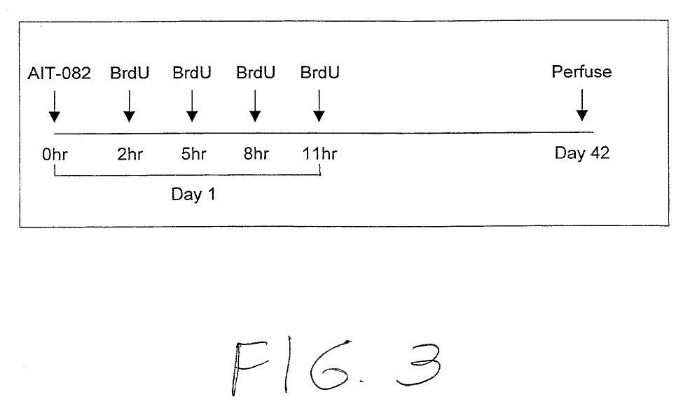

AIT-082 was administered intraperitoneally to adult mice (5-7 per group) at a dose of 1 -100 mg/kg. Saline was given as a negative control. Starting two hours after AIT-082 administration, animals received four intraperitoneal injections of bromodeoxyuridine (BrdU; 50 mg/kg each) at 3 hr intervals. Animals were perfused at 42 days after the AIT-082 administration. During these 42 days newly born neural stem cells have enough time to differentiate into neurons, astrocytes and other brain cells. This treatment regimen is outlined in FIG. 3.

Animals were perfused, brains treated and sections prepared as described for Example 1.

Every sixth section through the rostral-caudal extent of the hippocampus was immunostained with rat anti-BrdU paired with a Alexa 568 goat anti-rat IgG (red), mouse anti-NeuN (a neuronal marker) paired with Alexa 488 goat anti-mouse IgG (green), and rabbit anti-S100β (an astrocytes marker) paired with Alexa 647 goat anti-rabbit IgG (blue). Stereological techniques were used to estimate the total number of BrdU-positive nuclei in the dentate gyrus and the number of BrdU-positive cells that were also positive for NeuN (i.e indicating newly formed neurons) or S100β (indicating newly formed astrocytes). Cells that were BrdU-positive and not positive for either NeuN or S100β were labeled “other”. These cells may be undifferentiated neurons or other brain cells such as oligodendrocytes.

Results

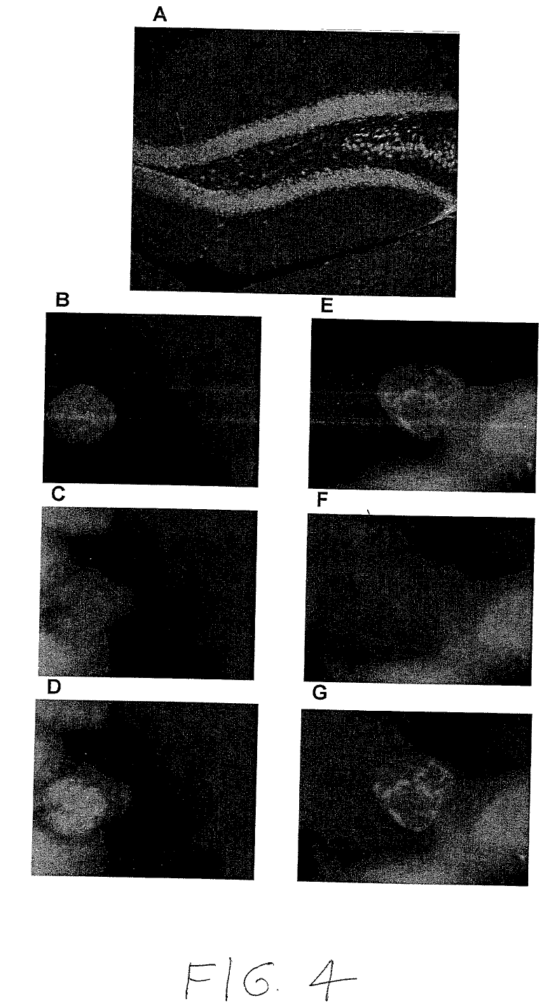

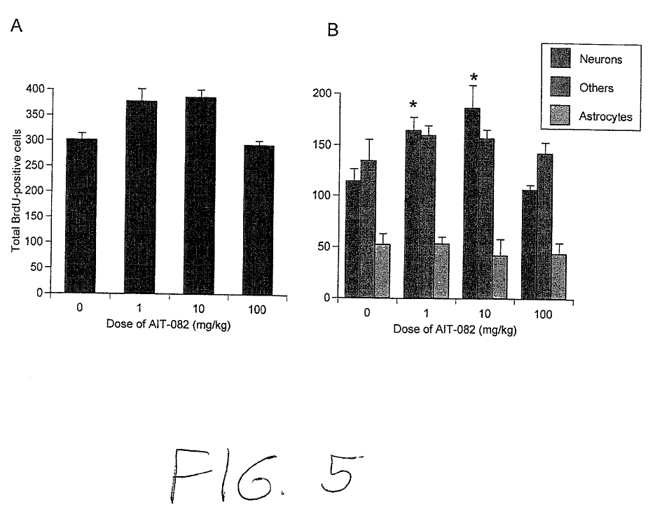

FIG. 4 is a series of photomicrographs of the dentate gyrus showing immunofluorescent colocalization of cell markers (A: immunofluorescent labeling of the dentate gyrus; B-D: colocalization of BrdU and NeuN; E-G: colocalization of BrdU and S100β). As seen in Example 1, treatment with 1 or 10 mg/kg AIT-082 resulted in an increase in the number of BrdU-positive cells (FIG. 5A). Furthermore, AIT-082 treatment caused a significant increase in the number of new neurons formed in the dentate gyrus (FIG. 5B).

Conclusion

Thus a single dose of AIT-082 increased the number of newly formed cells in the dentate gyrus in mice and these cells differentiated into neurons. Taken together with the data shown in Example 1, we conclude that, AIT-082 increases neurogenesis in mice.

### ADVANTAGES OF THE INVENTION

The present invention provides an efficient method of stimulating neurogenesis. The methods of the present invention can be used to treat a number of conditions or diseases in which neurons have died or their functioning has been impaired. The method is of particular use in treating Alzheimer's disease, Parkinson's disease, amyotrophic lateral sclerosis, stroke, multiple sclerosis, and other diseases and conditions. Methods according to the present invention can be used alone or together with other therapies. Methods according to the present invention also do not interfere with other treatments.

Although the present invention has been described in considerable detail, with reference to certain preferred versions thereof, other versions and embodiments are possible. Therefore, the scope of the invention is determined by the following claims.

While the specification describes particular embodiments of the present invention, those of ordinary skill can devise variations of the present invention without departing from the inventive concept.

I claim:

1. A method of inducing neurogenesis, where neurogenesis includes proliferation, differentiation, and/or survival of neural stem and progenitor cells, comprising administering to a mammal an effective amount of a compound having activity in inducing neurogenesis, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L wherein B is -OZ or N(Y₁)-D, where Z is hydrogen, alkyl, aryl, heteroaryl, cycloalkyl, aralkyl, or heteroaralkyl; D is a moiety that promotes absorption of the compound having activity in inducing neurogenesis; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S or an effective amount of a salt or prodrug ester of the compound having activity in inducing neurogenesis.

2. The method of claim 1 wherein the compound or the salt or prodrug ester of the compound having activity in inducing neurogenesis passes through the blood-brain barrier.

3. The method of claim 1 wherein A is a purine moiety.

4. The method of claim 3 wherein A is a substituted or unsubstituted hypoxanthine moiety.

5. The method of claim 4 wherein L has the structure —(CH₂)ₙ—CONH— where n is an integer from 1 to 6.

6. The method of claim 5 wherein the compound having activity in inducing neurogenesis is a compound of formula (I) 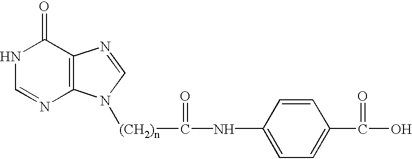
where n is an integer from 1 to 6 and R is hydrogen or lower alkyl.

7. The method of claim 5 wherein the compound having activity in inducing neurogenesis is a compound of formula (II) 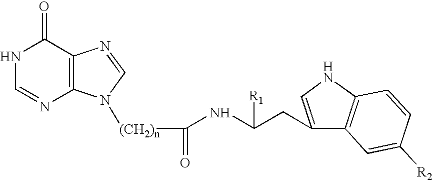
wherein n is an integer from 1 to 6, R is selected from the group consisting of H, COOH, and COOW₁, wherein W₁ is selected from the group consisting of lower alkyl, amino, and lower alkylamino, and R₂ is selected from the group consisting of H and OH.

8. The method of claim 5 wherein the compound having activity in inducing neurogenesis is a compound of formula (III) 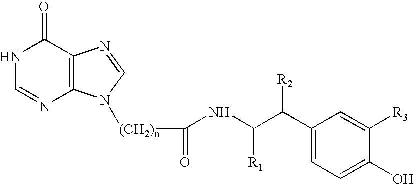
wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting from the group consisting of H and OH.

9. The method of claim 3 wherein A is a substituted or unsubstituted guanine moiety.

10. The method of claim 15 wherein L has the structure —(CH₂)ₙ—CONH— wherein n is an integer from 1 to 6.

11. The method of claim 10 wherein the compound having activity in inducing neurogenesis is a compound of formula (IV) 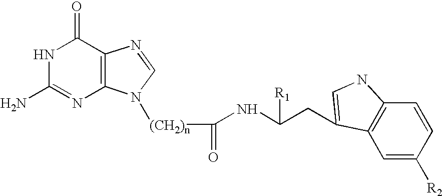
wherein n is an integer from 1 to 6, R₁ is selected from the group consisting of H, COOH, and COOW₁, wherein W₁ is selected from the group consisting of lower alkyl, amino, and lower alkylamino and R₂ is selected from the group consisting of H and OH.

12. The method of claim 10 wherein the compound having activity in inducing neurogenesis is a compound of formula (V) 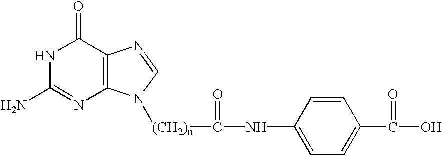
wherein n is an integer from 1 to 6 and R is selected from the group consisting of hydrogen and lower alkyl.

13. The method of claim 10 wherein the compound having activity in inducing neurogenesis is a compound of formula (VI) 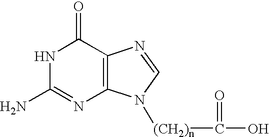
wherein n is an integer from 1 to 6 and R is selected from the group consisting of hydrogen and lower alkyl.

14. The method of claim 10 wherein the compound having activity in inducing neurogenesis is a compound of formula (VII) 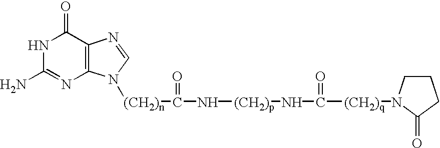
wherein n is an integer from 1 to 6, p is an integer from 1 to 6, and q is an integer from 1 to 3.

15. The method of claim 10 wherein the compound having activity in inducing neurogenesis is a compound of formula (VIII) 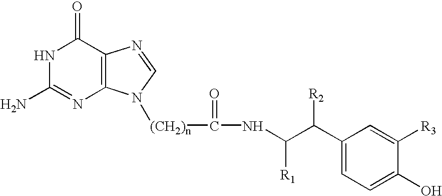
wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting of H and OH.

16. The method of claim 10 wherein the compound having activity in inducing neurogenesis is a compound of formula (IX) 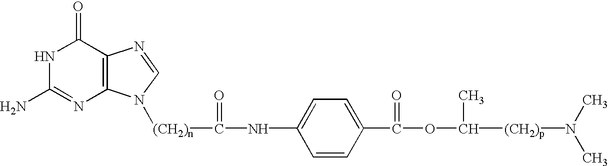
wherein n is an integer from 1 to 6 and p is an integer from 1 to 3.

17. The method of claim 1 wherein A is a substituted or unsubstituted 9-atom bicyclic moiety in which the 5-membered ring has 1 to 3 nitrogen atoms, the bicyclic moiety having the structure of formula (X) 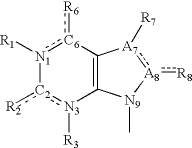
where:
(a) if the bond between N₁ and the bond between C₅ is a single bond, then the bond between C₆ and R₆ is a double bond, R₆ is O or S, and R₁ is hydrogen, alkyl, aralkyl, cycloalkyl, or heteroaralkyl;
(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(c) if the bond between C₂ and N₃ is a single bond, then the bond between C₂ and R₂ is a double bond, R₂ is O or S, and R₃ is hydrogen or alkyl;
(d) if the bond between C₂ and N₃ is a double bond, then the bond between C₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(e) A₇ and A₈ are C or N;
(i) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;
(ii) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;
(iii) if A₇ and A₈ are both N, then the bond between A₇ and A₈ is a double bond, and R₇ and R₈ are not present;
(iv) if A₇ is C and A₈ is N, then the bond between A₇ and A₈ is a double bond, R₇ is hydrogen, and R₈ is not present;
(v) if A₇ is N, A₈ is C, and the bond between A₇ and A₈ is a double bond, then R₇ is not present, the bond between A₈ is a single bond, and R₈ is hydrogen, halo, alkyl, alkenyl, aryl, aralkyl, aralkenyl, heteroaryl, heteroaralkyl, or heteroaralkenyl;
(vi) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S; and
(f) N₉ is bonded to L; with the proviso that A does not have the structure of an unsubstituted guanine or hypoxanthine.

18. The method of claim 3 wherein the purine moiety is a purine moiety of formula (XI) 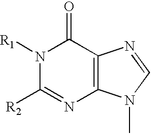
in which:
(a) R₁ is selected from the group consisting of hydrogen, alkyl, aralkyl, cycloalkyl, and heteroaralkyl; and R₂ is selected from the group consisting of hydrogen, alkyl, aralkyl, cycloalkyl, heteroaralkyl, halo, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroarylkylaminocarbonyl in which the alkyl portions could be cyclic and can contain from one to three heteroatoms which could be N, O, or S, with the proviso that both R₁ and R₂ are not hydrogen and that R₁ is not hydrogen when R₂ is amino.

19. The method of claim 3 wherein the purine moiety is a purine moiety of Formula (XII) 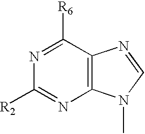
in which:
(a) R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃ where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaryloxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
(b) R₆ is selected from the group consisting of hydrogen, halo, amino, OQ₅, SQ₅, NHNH₂, NHOQ₅, NQ₅Q₆, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, aryisulfonyl, heteroarylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl in which the alkyl portions can be cyclic and can contain from 1 to 3 heteroatoms which can be N, O, or S.

20. The method of claim 3 wherein the purine moiety is the purine moiety of Formula (XIII) 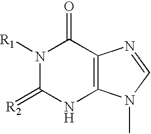
in which:
(a) R₁ is hydrogen, alkyl, aralkyl, cycloalkyl, or heteroaralkyl; and
(b) R₂is O or S.

21. The method of claim 1 wherein the compound having activity in inducing neurogenesis is a tetrahydroindolone derivative or analogue where A is a 9-atom bicyclic moiety in which the 5-membered ring has one to three nitrogen atoms, the bicyclic moiety being of Formula (XIV) 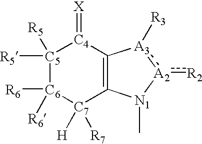
where:
(a) N₁ is bonded to L;
(b) A₂ and A₃ are C or N;
(i) if A₂ and A₃ are both C and the bond between A₂ and A₃ is a single bond, then the bond between A₂ and R₂ is two single bonds, two hydrogen atoms or is a double bond in which R₂ is O or S and R₃ is two hydrogen atoms;
(ii) if A₂ and A₃ are both C and the bond between A₂ and A₃ is a double bond, then R₃ is hydrogen, the bond between A₂ and R₂ is a single bond and R₂ is hydrogen, halo, alkyl, alkenyl, aryl, aralkyl, aralkenyl, heteroaryl, heteroaralkyl, or heteroaralkenyl;
(iii) if A₂ and A₃ are both N, then the bond between A₂ and A₃ is a double bond and R₂ and R₃ are not present;
(iv) if A₂ is N and A₃ is C, then the bond between A₂ and A₃ is a double bond, R₂ is not present, and R₃ is hydrogen;
(v) if A₂ is C, A₃ is N, and the bond between A₂ and A₃ is a double bond, then R₃ is not present, the bond between A₂ and R₂ is a single bond, and R₂ is hydrogen, halo, alkyl, alkenyl, aryl, aralkyl, aralkenyl, heteroaryl, heteroaralkyl, or heteroaralkenyl;
(vi) if A₂ is C, A₃ is N, and the bond between A₂ and A₃ is a single bond, then R₃ is hydrogen, alkyl, aryl, aralkyl, heteroaryl, or heteroaralkenyl, the bond between A₂ and R₂ is a double bond, and A₂ is O or S;
(c) R₅ is hydrogen, alkyl, aryl, aralkyl, heteroaryl, heteroaralkyl, alkanoyl, aroyl, heteroaroyl, aralkanoyl, heteroaralkanoyl, NH₂, NHQ₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(d) R_{5′} is hydrogen unless R₅ is alkyl, in which case R₅ is hydrogen or the same alkyl as R₅;
(e) R₅ and R_{5′} can be taken together as a double bond to C₅, and can be O, S, NQ₃, or C which can be substituted with one or two groups R₅, where Q₃ is alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S;
(f) R₆ is hydrogen, alkyl, aryl, aralkyl, heteroaryl, heteroaralkyl, NH₂, NHQ₄, NQ₄Q₅, OH, OQ₄, or SQ₄, where Q₄ and Q₅ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₄ and Q₅ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(g) R_{6′} is hydrogen unless R₆ is alkyl, in which case R_{6′} is hydrogen or the same alkyl as R₆;
(h) R₆ and R_{6′} can be taken together as a double bond to C₆ and can be O, S, NQ₆, or C which can be substituted with one or two groups R₅, and where Q₆ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S;
(i) R₇ is hydrogen unless R₅ is alkyl and R_{5′} is hydrogen, in which case R₇ is the same alkyl as R_{5·}; and
(j) X is oxygen, sulfur, or NH.

22. The method of claim 21 wherein A is a tetrahydroindolone moiety.

23. The method of claim 22 wherein the tetrahydroindolone moiety is a tetrahydroindolone moiety of formula (XV) 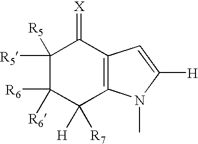
in which:
(a) R₅ is hydrogen, alkyl, aryl, aralkyl, heteroaryl, heteroaralkyl, alkanoyl, aroyl, heteroaroyl, aralkanoyl, heteroaralkanoyl, NH₂, NH₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S;
(b) R_{5′} is hydrogen;
(c) R₆ is hydrogen, alkyl, aryl, aralkyl, heteroaryl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S and where W₁ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from one to three heteroatoms which can be N, O, or S;
(d) R· is hydrogen;
(e) R₇ is hydrogen; and
(f) X is oxygen, sulfur, or NH.

24. The method of claim 1 wherein A is an amino-substituted 6-membered heterocyclic moiety of formula (XVI) 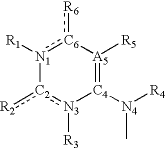
where:
(a) if the bond between N₁ and the bond between C₆ is a single bond, then the bond between C₆ and R₆ is a double bond, R₆ is O or S, and R₁ is hydrogen, alkyl, aralkyl, cycloalkyl, or heteroaralkyl;
(b) if the bond between N₁ and C₆ is a double bond, then the bond between C₆ and R₆ is a single bond, R₁ is not present, and R₆ is hydrogen, halo, amino, OH, OQ₁, SQ₁, NHNH₂, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(c) if the bond between C₂ and N₃ is a single bond, then the bond between C₂ and R₂ is a double bond, R₂ is O or S, and R₃ is hydrogen or alkyl;
(d) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OH, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(e) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;
(f) A₅ is carbon or nitrogen;
(g) if A₅ is nitrogen, then R₅ is not present;
(h) if A₅ is carbon, then R₅ is hydrogen, amino, alkyl, alkoxy, halo, nitro, aryl, cyano, alkenyl, or alkaryl;
(i) if R₅ and R₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
(j) N₄ is bonded to L.

25. The method of claim 24 wherein A₅ is carbon and the 6-membered heterocyclic moiety is a pyrimidine moiety.

26. The method of claim 25 wherein R₂ is O and R₃ is hydrogen.

27. The method of claim 26 wherein the pyrimidine moiety is selected from the group consisting of cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, and 5-hydroxymethyluracil.

28. The method of claim 25 wherein R₂ is S and R₃ is hydrogen.

29. The method of claim 28 wherein the pyrimidine moiety is selected from the group consisting of 2-thiouracil, 5-methylamino-2-thiouracil, 5-methyl-2-thiouracil, and 2-thiocytosine.

30. The method of claim 25 wherein R₂ is amino and the bond between C₂ and N₃ is a double bond.

31. The method of claim 30 wherein the pyrimidine moiety is selected from the group consisting of 2-aminopyrimidinone and 2-amino-4-chloropyrimidine.

32. The method of claim 25 wherein R₂ is hydrogen and the bond between C₂ and N₃ is a double bond.

33. The method of claim 32 wherein the pyrimidine moiety is selected from the group consisting of 4-chloropyrimidine, 5-amino-4-chloropyrimidine, 4-chloro-5-methylpyrimidine, 4-chloro-5-hydroxymethylpyrimidine, and 4-chloro-5-carboxymethylpyrimidine.

34. The method of claim 25 wherein R₁ is hydrogen, methyl, or ethyl, R₅ is hydrogen, methyl, or ethyl, and R₆ is O.

35. The method of claim 34 wherein the pyrimidine moiety is pyrimidinone.

36. The method of claim 1 wherein L has the structure —(CH₂)ₙ— wherein n is an integer from 1 to 6.

37. The method of claim 1 wherein the moiety B is -OZ.

38. The method of claim 37 wherein Z is hydrogen.

39. The method of claim 37 wherein Z is alkyl.

40. The method of claim 39 wherein Z is selected from the group consisting of methyl, ethyl, butyl, propyl, and isopropyl.

41. The method of claim 1 wherein B is —N(Y₁)-D.

42. The method of claim 41 wherein Y₁ is hydrogen.

43. The method of claim 42 wherein Y₁ is lower alkyl.

44. The method of claim 43 wherein Y₁ is methyl.

45. The method of claim 41 wherein D is a moiety having at least one polar, charged, or hydrogen-bond-forming group to increase the water-solubility of the compound having activity in inducing neurogenesis.

46. The method of claim 45 wherein D is a carboxylic acid or carboxylic acid ester with the structure 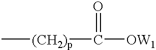
wherein p is an integer from 1 to 6 and W₁ is selected from the group consisting of hydrogen and lower alkyl.

47. The method of claim 46 wherein W₁ is hydrogen.

48. The method of claim 46 wherein W₁ is ethyl.

49. The method of claim 45 wherein D and Y₁ are taken together to form a piperazine derivative of the structure 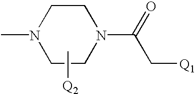
wherein Q₁ is hydrogen, methyl, ethyl, butyl, or propyl, and Q₂ is hydrogen or methyl, where, if Q₂ is methyl, it can be located on either of the two possible positions in the piperazine ring.

50. The method of claim 45 wherein D has the structure 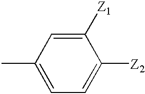
wherein one of Z₁ and Z₂ is hydrogen and the other is Z₁ and Z₂ is —COOH or —COOW₁, wherein W₁ is alkyl.

51. The method of claim 50 wherein W₁ is selected from the group consisting of methyl, ethyl, propyl, butyl, and isobutyl.

52. The method of claim 45 wherein D is a phenylsulfonamidyl moiety of the structure 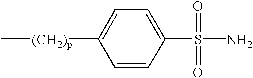
wherein p is an integer from 0 to 6.

53. The method of claim 45 wherein D is an alkylpyridyl moiety of the structure 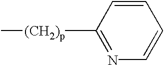
wherein p is an integer from 1 to 6.

54. The method of claim 45 wherein D is a dialkylaminoalkyl moiety of the structure 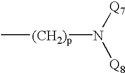
wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S, and when Q₇ and Q₈ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

55. The method of claim 45 wherein Q₇ and Q₈ are each alkyl.

56. The method of claim 55 wherein Q₇ and Q₈ are each selected from the group consisting of methyl, ethyl, propyl, butyl, and isobutyl.

57. The method of claim 55 wherein Q₇ and Q₈ are taken together to form a 5- or 6-membered optionally substituted ring.

58. The method of claim 57 wherein the ring is a morpholinyl ring.

59. The method of claim 57 wherein the ring is a pyrrolidinyl ring that is optionally substituted with oxo.

60. The method of claim 57 wherein the ring is a piperidinyl ring that is optionally substituted with methyl or ethyl.

61. The method of claim 45 wherein D is an alkylpyrrolidinyl moiety of the structure 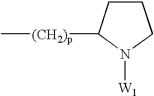
wherein p is an integer from 1 to 6 and W₁ is selected from the group consisting of methyl, ethyl, and propyl.

62. The method of claim 1 wherein the mammal is an adult mammal.

63. The method of claim 1 wherein the neural stem and progenitor cells are selected from the group consisting of the neural stem and progenitor cells present in the dentate gyrus of the hippocampus and the neural stem and progenitor cells present in the subventricular zone.

64. The method of claim 63 wherein the neural stem and progenitor cells are the neural stem and progenitor cells present the dentate gyrus of the hippocampus.

65. The method of claim 63 wherein the neural stem and progenitor cells are the neural stem and progenitor cells present in the subventricular zone.

66. A method of inducing neurogenesis comprising administering to a mammal an effective amount of N-4-[[3-(6-oxo-1,6-dihydropurin-9-yl)-1-oxopropyl] amino] benzoic acid.
